(19) United States
(12) Patent Application Publication
(10) Pub. No.: US 2014/0287071 A1
(43) Pub. Date: Sep. 25, 2014

(54) VETERINARY SUPPLEMENTS

(71) Applicant: LifeVantage Corporation, Sandy, UT (US)

(72) Inventor: William M. Barnett, III, Temecula, CA (US)

(73) Assignee: LifeVantage Corporation, Sandy, UT (US)

(21) Appl. No.: 14/213,988

(22) Filed: Mar. 14, 2014

Related U.S. Application Data
(60) Provisional application No. 61/753,544, filed on Jan. 17, 2013.

(51) Int. Cl. A61K 38/39; A61K 31/202; A61K 36/82; A61K 36/9066; A61K 36/28; A61K 36/81

(52) U.S. Cl. CPC A61K 38/39; A61K 36/28; A61K 36/81; A61K 36/82; A61K 36/9066; A61K 31/202
USPC 424/729

(57) ABSTRACT

Veterinary supplements comprising one or more Nrf2-activating agents are disclosed. The veterinary supplements may further comprise omega-3 fatty acids and collagen. The veterinary supplements are effective for treating, inhibiting, reducing and/or preventing oxidative stress.

### CROSS-REFERENCE TO RELATED APPLICATIONS

This application claims the benefit under 35 U.S.C. §119(e) of U.S. Provisional Application No. 61/753,544 filed Jan. 17, 2013, the entire contents of which are incorporated herein by reference.

### BACKGROUND OF THE INVENTION

Oxidative stress is a condition characterized by elevated levels of free radicals and reactive oxygen species in the blood stream. Oxidative stress occurs in a variety of animals, and is associated with a variety of diseases and conditions, including inflammation, joint disorders, arthritis, cognitive dysfunction, diabetes, pancreatitis, respiratory diseases, and cardiac and vision disorders. Symptoms of oxidative stress in animals include disorientation, decreased social interaction, loss of prior house training, sleep disturbance, and decreased mobility. Oxidative stress is also thought to contribute to certain illnesses, such as viral infections, including feline immunodeficiency virus, and to genetically predisposed conditions, including canine hip dysplasia and asthma. Accordingly, a need exists for veterinary supplements that treat or prevent oxidative stress and the associated diseases and conditions.

Nuclear factor erythroid-2-related factor 2 (Nrf2) is a transcription factor that plays a central role in the Nrf2 antioxidant response pathway. Nrf2 regulates the expression of several antioxidant enzymes. Under normal conditions, Nrf2 is kept in the cytoplasm by a cluster of proteins that degrade it quickly. Under oxidative stress, Nrf2 is not degraded, but instead travels to the nucleus where it binds to DNA and activates transcription of antioxidative and cytoprotective genes and ultimately their protein products, which enable cells to survive in the face of stress from free radicals and other antioxidants. Nrf2 also down-regulates other genes that promote inflammation and fibrosis.

### SUMMARY OF THE INVENTION

Disclosed herein are veterinary supplements comprising one or more Nrf2-activating agents. In some embodiments, the veterinary supplements further comprise omega-3 fatty acids. In some embodiments, the veterinary supplements further comprise collagen. In some embodiments, the veterinary supplements further comprise omega-3 fatty acids and collagen.

In some embodiments, the one or more Nrf2-activating agents are selected from the group consisting of Bacopa extract, milk thistle extract, Ashwagandha extract, green tea extract, turmeric extract, Gotu kola powder, Aloe vera powder, Gingko biloba leaf extract, N-Acetyl Cysteine, piperine, resveratrol, pterostilbene, sulfurophane, ginger, cinnamon, wasabi, carnosic acid, lipoic acid, licorice, lycopene, and combinations thereof.

In some embodiments, the one or more Nrf2-activating agents are selected from the group consisting of Bacopa extract, milk thistle extract, Ashwagandha extract, green tea extract, turmeric extract, and combinations thereof.

In some embodiments, the one or more Nrf2-activating agents comprises Bacopa extract, milk thistle extract, Ashwagandha powder, green tea extract, and turmeric extract.

In some embodiments, the veterinary supplements comprise Type II chicken sternum collagen.

In some embodiments, the one or more Nrf2-activating agents is present in an amount of from about 100 mg to about 200 mg, said omega-3 fatty acids are present in an amount of about 100 mg to about 300 mg, and said collagen is present in an amount of from about 75 mg to about 175 mg. In some embodiments, the veterinary supplements of embodiment 4, wherein said one or more Nrf2-activating agents is present in an amount of from about 125 mg to about 175 mg, said omega-3 fatty acids are present in an amount of about 150 mg to about 250 mg, and said collagen is present in an amount of from about 100 mg to about 150 mg. In some embodiments, the one or more Nrf2-activating agents is present in an amount of about 150 mg, said omega-3 fatty acids are present in an amount of about 200 mg, and said collagen is present in an amount of about 125 mg.

In some embodiments, the veterinary supplements comprise at least about 50 mg milk thistle extract, at least about 33.33 mg Ashwagandha extract, at least about 16.67 mg green tea extract, at least about 33.33 mg B. monniera extract, at least about 16.67 mg Turmeric extract, at least about 200 mg omega-3 fatty acids, and at least about 125 mg of collagen.

In some embodiments, the veterinary supplements comprise about 50 mg milk thistle extract, about 33.33 mg Ashwagandha extract, about 16.67 mg green tea extract, about 33.33 mg B. monniera extract, about 16.67 mg Turmeric extract, about 200 mg omega-3 fatty acids, and about 125 mg of collagen.

In some embodiments, the veterinary supplements comprise inactive ingredients selected from the group consisting of dicalcium phosphate, hydoxypropyl cellulose, hydroxylpropyl methylcellulose, magnesium stearate, maltodextrin, microcrystalline cellulose, silicon dioxide, stearic acid, sucrose fatty acid ester, chicken flavoring, chicken liver flavoring, smoked bacon flavoring, and combinations thereof.

Disclosed herein are methods of administering a veterinary supplement comprising one or more Nrf2-activating agents to an animal. In some embodiments, the veterinary supplements further comprise omega-3 fatty acids. In some embodiments, the veterinary supplements further comprise collagen. In some embodiments, the veterinary supplements further comprise omega-3 fatty acids and collagen.

In some embodiments, the methods disclosed herein treat, inhibit, reduce, and/or prevent oxidative stress. In some embodiments, the method treats, inhibits, reduces, and/or prevents conditions associated with oxidative stress. In some embodiments, the method treats, inhibits, reduces, and/or prevents conditions associated with oxidative stress are selected from the group consisting of inflammation, joint disorders, arthritis, cognitive dysfunction, diabetes, pancreatitis, respiratory diseases, cardiac disorders, vision disorders, and combinations thereof. In some embodiments, the method supports joint function, improves mobility and flexibility, enhances cognitive function, and combinations thereof.

### BRIEF DESCRIPTION OF THE DRAWINGS

FIG. 1 is a chart showing Owner Overall Disability Score in dogs with hip disabilities over 60 days.

FIG. 2 is a chart showing % average changes in catalase activity (U) from baseline in active and placebo groups.

### DETAILED DESCRIPTION OF THE INVENTION

Disclosed herein are veterinary supplements for treating, inhibiting, reducing, and/or preventing oxidative stress. In some embodiments, the veterinary supplements disclosed herein treat, inhibit, reduce, and/or prevent conditions associated with oxidative stress, including but not limited to inflammation, joint disorders, arthritis, cognitive dysfunction, diabetes, pancreatitis, respiratory diseases, and cardiac and vision disorders. In some embodiments, veterinary supplements disclosed herein support joint function, improve mobility and flexibility, enhance cognitive function, and combinations thereof.

The veterinary supplements disclosed herein may be used in any suitable animals, including domestic animals (e.g., dogs, cats, and the like), farm animals (e.g., cows, sheep, pigs, horses, and the like), and laboratory animals (e.g., rats, mice, guinea pigs, and the like). The veterinary supplements disclosed herein include optimal dosages for the particular patient animal. For example, in some embodiments, the dosage is optimal for the specific weight or weight range of the patient animal species.

In some embodiments, the veterinary supplements disclosed herein comprise Nrf2-activating agents. As used herein, a “Nrf2-activating agent” is a chemical compound, biological molecule, composition, formulation, and/or extract that activates transcription of antioxidative and/or cytoprotective genes through the Nrf2 antioxidant response pathway.

In certain embodiments, the veterinary supplements disclosed herein comprise Nrf2-activating agents in an effective amount. An “effective amount” is a quantity provided by a particular route of administration and dosing regimen that is sufficient to achieve a desired therapeutic, inhibitory, and/or prophylactic effect. For example, an effective amount of a Nrf2-activating agent is a quantity provided by a particular route of administration and dosing regimen that is sufficient to activate transcription of antioxidative and cytoprotective genes through the Nrf2 antioxidant response pathway and treat, inhibit, reduce, and/or prevent oxidative stress in the patient animal.

As used herein, the term “about,” when located before a dosage amount or dosage range of a specific ingredient, refers to an amount or range closely above and/or closely below the stated amount or range that does not manifestly alter the therapeutic effect of the specific ingredient from the stated amount or range and is meant to encompass at least all equivalents of that amount. In some specific embodiments, the term “about,” when located before a dosage amount or dosage range of a specific ingredient, refers to an amount or range that is +10% of the stated amount or range. Numerical quantities given herein are approximate unless stated otherwise, meaning that the term “about” can be inferred when not expressly stated.

In some specific embodiments, an effective amount of a Nrf-2 activating agent is from about 25 mg to about 1,000 mg, or from about 50 mg to about 750 mg, or from about 75 mg to about 650 mg, or from about 100 mg to about 500 mg, or from about 100 mg to about 200 mg, or from about 125 mg to about 175 mg. In certain specific embodiments, an effective amount of a Nrf-2 activating agent is about 25 mg, about 50 mg, about 75 mg, about 100 mg, about 125 mg, about 150 mg, about 175 mg, about 200 mg, about 225 mg, about 250 mg, about 275 mg, about 300 mg, about 350 mg, about 400 mg, about 450 mg, about 500 mg, about 750 mg, or about 1,000 mg. In a specific embodiment, an effective amount of a Nrf-2 activating agent is at least about 150 mg.

In certain embodiments, Nrf2-activating agents include plant extracts or powders. Suitable Nrf2-activating agents for use in the veterinary supplements disclosed herein include Bacopa extract, milk thistle extract, Ashwagandha powder, green tea extract, turmeric extract, Gotu kola powder, Aloe vera powder, Gingko biloba leaf extract, N-Acetyl Cysteine, piperine, resveratrol, pterostilbene, sulfurophane, ginger, cinnamon, wasabi, carnosic acid, lipoic acid, licorice, lycopene, and combinations thereof.

Bacopa monniera

Bacopa monniera (common names: water hyssop and Brahmi) is a creeping perennial that thrives in warmer temperate climates. The genus Bacopa includes over 100 species of aquatic herbs distributed throughout the warmer regions of the world. The plant is a profusely branched herb, rooting at the nodes and forming dense mats. B. monniera extract (Bacopin®) is a standardized extract prepared from the leaves of the B. monniera plant (Sabinsa Corporation, Piscataway, N.J., USA). In some embodiments, it is standardized for a minimum of 20% bacosides A & B. Other extracts of the B. monniera plant standardized for greater minimum levels of bacosides A & B (e.g., 30%, 40%, 50%, etc.) are useful in the veterinary supplements disclosed herein and can be prepared by extraction techniques known in the art. Extract of B. monniera is commercially available, e.g., Viable Herbal Solutions (Morrisville, Pa., USA).

In some embodiments, the veterinary supplements disclosed herein comprise from about 1 mg to about 1,000 mg B. monniera extract, or from about 10 mg to about 500 mg B. monniera extract, or from about 20 mg to about 100 mg B. monniera extract. In a specific embodiment, at least about 33.33 mg B. monniera extract is contained in the veterinary supplements disclosed herein. In a specific embodiment, about 33.33 mg B. monniera extract is contained in the veterinary supplements disclosed herein. In certain embodiments, the B. monniera extract of the herb-containing composition is Bacopin®.

In a specific embodiment, veterinary supplements disclosed herein contain a B. monniera extract standardized for at least about 20% bacosides A & B. In another specific embodiment, veterinary supplements disclosed herein contain a B. monniera extract standardized for at least about 30% bacosides A & B. In another specific embodiment, veterinary supplements disclosed herein contain a B. monniera extract standardized for at least about 40% bacosides A & B. In another specific embodiment, veterinary supplements disclosed herein contain a B. monniera extract standardized for at least about 50% bacosides A & B. In another specific embodiment, veterinary supplements disclosed herein contain a B. monniera extract standardized for about 50% bacosides A & B.

Milk Thistle

Milk thistle (botanical name; Silybum marianum; other common names: Marian, Silybum, Silymarin) is a fine, tall plant, about the size of the Cotton Thistle, with cut into root-leaves, waved and spiny at the margin, of a deep, glossy green, with milk white veins, and is found not uncommonly in hedgebanks and on waste ground. Useful parts of the plant include, e.g., the whole herb, root, leaves, seeds, and hull. Milk thistle seeds contain a bioflavonoid complex known as silymarin. Silymarin is an extract of the seeds of the milk thistle plant. In some embodiments, a standardized extract should be 80% silymarin (the active ingredient). Silymarin is made up of three parts: silibinin, silidianin, and silicristin. Milk thistle (80% silymarin) extract is commercially available, e.g., Stayleaner.com (Las Vegas, Nev., USA).

In some embodiments, the veterinary supplements disclosed herein comprise from about 15 mg to about 2,000 mg milk thistle extract (70%-80% silymarin), or from about 25 mg to about 500 mg milk thistle extract (70%-80% silymarin), or from about 40 mg to about 150 mg milk thistle extract (70%-80% silymarin). In a specific embodiment the veterinary supplements disclosed herein comprise at least about 50 mg milk thistle extract (70%-80% silymarin). In a specific embodiment the veterinary supplements disclosed herein comprise about 50 mg milk thistle extract (70%-80% silymarin).

In some embodiments, the veterinary supplements disclosed herein comprise from about 15 mg to about 2,000 mg milk thistle extract, or from about 25 mg to about 500 mg milk thistle extract, or from about 40 mg to about 150 mg milk thistle extract. In a specific embodiment the veterinary supplements disclosed herein comprise at least about 50 mg milk thistle extract. In a specific embodiment the veterinary supplements disclosed herein comprise about 50 mg milk thistle extract.

Ashwagandha

Ashwagandha (botanical names: Withania somnifera and Physalis flexuosa; other common names: winter cherry, Ashgandh, Achuvagandi, Amikkira-gadday, Amkulang-kalang, Amukkira-kilzhangu, Amukran-kizhangu, Asagandha, Asana, Asgandh, Asundha, Asvagandhi, Fatarfoda, Hirimaddina-gadday, Hirre-gadday, Penneroo-gadda, Pevette, Sogade-beru, Indian ginseng) is an erect branched shrub native to India, Pakistan and Sri Lanka. Ashwaganda powder is commercially available, e.g., iHerb Inc. (Monrovia, Calif., USA).

In some embodiments, the veterinary supplements disclosed herein comprise from about 1 mg to about 1,000 mg Ashwagandha root extract, or from about 10 mg to about 500 mg Ashwagandha root extract, or from about 20 mg to about 100 mg Ashwagandha root extract. In a specific embodiment, the veterinary supplements disclosed herein comprise at least about 33.33 mg Ashwagandha root extract. In a specific embodiment, the veterinary supplements disclosed herein comprise about 33.33 mg Ashwagandha root extract.

In some embodiments, the veterinary supplements disclosed herein comprise from about 1 mg to about 1,000 mg Ashwagandha powder, or from about 10 mg to about 500 mg Ashwagandha powder, or from about 20 mg to about 100 mg Ashwagandha powder. In a specific embodiment, the veterinary supplements disclosed herein comprise at least about 33.33 mg Ashwagandha powder. In a specific embodiment, the veterinary supplements disclosed herein comprise about 33.33 mg Ashwagandha powder.

Turmeric

Turmeric extract 95% is prepared from the root or rhizome of the Curcuma longa plant (common names: Curcuma, Turmeric, Ukon, Goeratji, Kakoenji, Koenjet, Kondin, Kunir, Kunyit, Oendre, Rame, Renet, Temu kuning, Temu kunyit, Tius. Curcumin). C. longa is a perennial plant native to India. A compound called curcumin is an extract of the root. In some embodiments, turmeric extract that is standardized to 95% curcumin contains turmeric (with 95% curcumin). Turmeric extract 95% is commercially available, e.g., EZ-FITNESS (Northborough, Mass., USA).

In some embodiments, the veterinary supplements disclosed herein comprise from about 1 mg to about 1,000 mg Turmeric extract, or from about 10 mg to about 300 mg Turmeric extract, or from about 12.5 mg to about 100 mg Turmeric extract. In a specific embodiment, the veterinary supplements disclosed herein comprise, at least about 16.67 mg Turmeric extract. In a specific embodiment, the veterinary supplements disclosed herein comprise, about 16.67 mg Turmeric extract.

In some embodiments, the veterinary supplements disclosed herein comprise from about 1 mg to about 1,000 mg Turmeric extract (95% curcumin), or from about 10 mg to about 300 mg Turmeric extract (95% curcumin), or from about 12.5 mg to about 100 mg Turmeric extract (95% curcumin). In a specific embodiment, the veterinary supplements disclosed herein comprise, at least about 16.67 mg Turmeric extract (95% curcumin). In a specific embodiment, the veterinary supplements disclosed herein comprise, about 16.67 mg Turmeric extract (95% curcumin).

Gotu kola

Gotu kola (botanical names: Hydrocotyle asiatica, Centella asiatica; other common names: Centella, March Pennywort, Indian Pennywort, Hydrocotyle, Brahmi (Sanskrit), Luei Gong Gen (Chinese)) is a slender, creeping perennial plant that grows commonly in swampy areas of India, Sri Lanka, Madagascar, South Africa and the tropics. Gotu kola is distinct from the kola nut. Gotu kola powder is prepared from the leaves and aerial parts of the plant and used for medicinal purposes. Gotu kola powder is commercially available, e.g., @Internatural (Twin Lakes, Wis., USA).

In some embodiments, the veterinary supplements disclosed herein comprise, from about 10 mg to about 4,000 mg Gotu kola powder, or from about 25 mg to about 2,000 mg Gotu kola powder, or from about 50 mg to about 1,000 mg Gotu kola powder. In a specific embodiment, the veterinary supplements disclosed herein comprise at least about 200 mg Gotu kola powder. In a specific embodiment, the veterinary supplements disclosed herein comprise about 200 mg Gotu kola powder.

Aloe vera

Aloe vera (common names: medicinal aloe, burn plant, Barbados aloe, unguentine cactus) is a perennial plant. The strong, fibrous root produces a rosette of fleshy basal leaves as in the agave but considerably smaller that grows wild in East and South Africa and also cultivated in the West Indies and other tropical areas. Aloe vera powder is commercially available, e.g., Red Lion International Trading & Brokerage Co. (Fullerton, Calif., USA).

In some embodiments, the veterinary supplements disclosed herein comprise from about 10 mg to about 4,000 mg Aloe vera powder, or from about 25 mg to about 2,000 mg Aloe vera powder, or from about 50 mg to about 1,000 mg Aloe vera powder. In a specific embodiment, the veterinary supplements disclosed herein comprise at least about 200 mg Aloe vera powder. In a specific embodiment, the veterinary supplements disclosed herein comprise about 200 mg Aloe vera powder.

Green Tea

Green tea extracts are useful in the compositions of the present invention. In some embodiments of the compositions of the invention, the green tea extract is standardized for polyphenols. For example, green tea extract, 98% polyphenols containing 45% polyphenols such as polyphenol (-)-epigallocatechin gallate (EGCG) is prepared from the leaf of the tea herb Camellia sinensis. Green tea extracts are commercially available, e.g., Hunan Kinglong Bio-Resource Co., Ltd., (Xingsha, Changsha, Hunan, P. R. China).

In some embodiments, the veterinary supplements disclosed herein comprise from about 1 mg to about 1,000 mg green tea extract, or from about 10 mg to about 300 mg green tea extract, or from about 12.5 mg to about 100 mg green tea extract. In a specific embodiment, the veterinary supplements disclosed herein comprise at least about 16.67 mg green tea extract. In a specific embodiment, the veterinary supplements disclosed herein comprise about 16.67 mg green tea extract.

In some embodiments, the veterinary supplements disclosed herein comprise from about 1 mg to about 1,000 mg green tea extract (98% polyphenols, 45% EGCG), or from about 10 mg to about 300 mg green tea extract (98% polyphenols, 45% EGCG), or from about 12.5 mg to about 100 mg green tea extract (98% polyphenols, 45% EGCG). In a specific embodiment, the veterinary supplements disclosed herein comprise at least about 16.67 mg green tea extract (98% polyphenols, 45% EGCG). In a specific embodiment, the veterinary supplements disclosed herein comprise about 16.67 mg green tea extract (98% polyphenols, 45% EGCG).

Gingko biloba

Ginkgo biloba (common name: Maidenhair tree) is a dioecious tree. Ginko biloba extract is commercially available, e.g., from iHerb Inc. (Monrovia, Calif., USA).

In some embodiments, the veterinary supplements disclosed herein comprise from about 5 mg to about 2,000 mg G. biloba leaf extract, or from about 10 mg to about 1,000 mg G. biloba leaf extract, or from about 50 mg to about 500 mg G. biloba leaf extract. In a specific embodiment, the veterinary supplements disclosed herein comprise at least about 200 mg G. biloba leaf extract. In a specific embodiment, the veterinary supplements disclosed herein comprise about 200 mg G. biloba leaf extract.

N-Acetyl Cysteine

N-Acetyl Cysteine (NAC) is an acetylated form of the amino acid cysteine. N-Acetyl Cystein is commercially available, e.g., Doctor's Trust Vitamins (Orlando, Fla., USA).

In some embodiments, the veterinary supplements disclosed herein comprise from about 50 mg to about 5,000 mg N-Acetyl Cysteine, or from about 100 mg to about 4,000 mg N-Acetyl Cysteine, or from about 250 mg to about 2,000 mg N-Acetyl cysteine. In a specific embodiment, the veterinary supplements disclosed herein comprise at least about 500 mg N-Acetyl Cysteine. In a specific embodiment, the veterinary supplements disclosed herein comprise about 500 mg N-Acetyl Cysteine.

Piperine

Piperine is an alkaloid that can be isolated from black pepper, white pepper, and long pepper. Chavicine is an isomer of piperine. Piperine is commercially available as an extract of black pepper (BioPerine®) or as the isolated compound (e.g., from Sigma-Aldrich®).

Resveratrol

Resveratrol is a stilbenoid and phytoalexin that is produced naturally by several plants, including grapes. Resveratrol is commercially available in various extracts or as the isolated compound (e.g., from Sigma-Aldrich®).

Pterostilbene

Pterostilbene is a stilbenoid and phytoalexin that is produced naturally by blueberries and grapes. Pterostilbene is commercially available in various extracts or as the isolated compound (e.g., from Sigma-Aldrich®).

Sulforaphane

Sulforaphane is an isothiocyanate-containing molecule that is found in cruciferous vegetables such as broccoli, Brussels sprouts, and cabbages. Sulforaphane is commercially available in various extracts or as the isolated compound (e.g., from Sigma-Aldrich®).

Ginger

Ginger is the rhizome of the plant Zingiber officinale. Various formulations and extracts of ginger are commercially available.

Cinnamon

Cinnamon, sometimes referred to specifically as “true cinnamon” or “cassia,” is a spice obtained from the inner bark of trees from the genus Cinnamomum, including Cinnamomum verum. Various formulations and extracts of cinnamon are commercially available.

Wasabi

Wasabi is a member of the Brassicaceae family of plants. Wasabi is available in powder and paste forms.

Carnosic Acid

Carnosic acid is a benzenediol abietane diterpene found in rosmarius officinalis and Salvia officinalis. Carnosic acid is commercially available in various plant extracts or as the isolated compound (e.g., from Sigma-Aldrich®).

Lipoic Acid

Lipoic acid (also known as a-lipoic acid) is found in many natural food sources, but is more prevalent in kidney, heart, liver, spinach, broccoli, and yeast extract. The amount of lipoic acid present is food sources is typically quite low, but the compound can be synthesized by methods known in the art. Purified lipoic acid is commercially available (e.g., from Sigma-Aldrich®).

Licorice

Licorice is the root of Glycyrrhiza glabra. Various extracts of licorice are commercially available.

Lycopene

Lycopene is a bright red carotenoid (carotene) pigment and phytochemical found in various red fruits and vegetables, including tomatoes, red carrots, red bell peppers, watermelons, gac, and papayas. Lycopene is available in various plant extracts (e.g., from Sigma-Aldrich®).

In some embodiments, veterinary supplements disclosed herein further comprise omega-3 fatty acids, collagen, or a combination thereof.

In some embodiments, omega-3 fatty acids include docosahexaenoic acid (DHA), eisocapentaenoic acid (EPA), a-linolenic acid (ALA), and combinations thereof. In some embodiments, the source for omega-3 fatty acids is a marine source, including but not limited to fish oils, algal oil, and squid oil. In certain embodiments, the source of omega-3 fatty acids is plant oils.

In certain embodiments, an effective amount of omega-3 fatty acids is an amount from about 25 mg to about 1,000 mg, or from about 50 mg to about 800 mg, or from about 75 mg to about 600 mg, from about 100 mg to about 300 mg, or from about 150 mg to about 250 mg. In certain specific embodiments, an effective amount of omega-3 fatty acids is about 25 mg, about 50 mg, about 75 mg, about 100 mg, about 150 mg, about 200 mg, about 250 mg, about 300 mg, about 350 mg, about 400 mg, about 450 mg, about 500 mg, about 750 mg, or about 1,000 mg. In a specific embodiment, an effective amount of omega-3 fatty acids is at least about 200 mg omega-3 fatty acids. In a specific embodiment, an effective amount of omega-3 fatty acids is about 200 mg omega-3 fatty acids.

In some embodiments, the source of collagen is an animal. In certain embodiments, the collagen is a source of natural glucosamine, chondroitin, and hyaluronic acids. In a specific embodiment, veterinary supplements disclosed herein include collagen in the form of Type II collagen, for example, Type II chicken sternum collagen.

In certain embodiments, an effective amount of collagen is an amount from about 25 mg to about 500 mg, or from about 50 mg to about 300 mg, from about 100 mg to about 300 mg, from about 75 mg to about 175 mg, or from about 100 mg to about 150 mg. In certain specific embodiments, an effective amount of collagen is about 25 mg, about 50 mg, about 75 mg, about 100 mg, about 125 mg, about 150 mg, about 175 mg, about 200 mg, about 250 mg, about 300 mg, about 350 mg, about 400 mg, about 450 mg, or about 500 mg. In a specific embodiment, an effective amount of collagen is at least about 125 mg of collagen. In a specific embodiment, an effective amount of collagen is about 125 mg of collagen.

The veterinary supplements disclosed herein may be administered in various therapeutic and/or prophylactic methods. For example, the veterinary supplements disclosed herein can be administered to an animal to treat, inhibit, reduce, and/or prevent conditions associated with oxidative stress, including but not limited to inflammation, joint disorders, arthritis, cognitive dysfunction, diabetes, pancreatitis, respiratory diseases, and cardiac and vision disorders. In some embodiments, veterinary supplements disclosed herein can be administered to an animal to support joint function, improve mobility and flexibility, enhance cognitive function, and combinations thereof.

The veterinary supplements disclosed herein may be administered in various dosage forms. The term “dosage form,” as used herein, is the form in which the dose is to be administered to the animal. Dosage forms, for example, may be solid, liquid or gaseous. Dosage forms may include, for example, a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution. In a specific embodiment, the dosage form is a chewable tablet.

In some embodiments, the veterinary supplements disclosed herein are prepared with inactive ingredients that will protect the active agents from rapid elimination from the body, such as a controlled release formulation, implants, or microencapsulated delivery system. Such systems may improve bioavailability and stability of the active agents. The dosage forms may be administered by any suitable means, including but not limited to orally, sublingually, nasally, topically, parenterally, or intravenously. The dosage forms may be administered as frequently as needed until the desired therapeutic or prophylactic effect is achieved, for example, once daily, twice daily, thrice daily, etc.

In some embodiments, the veterinary supplements disclosed herein may include biologically or pharmacologically active agents useful for treating, inhibiting, reducing, and/or preventing oxidative stress other than those specifically disclosed herein. For example, in certain specific embodiments, the veterinary supplements disclosed herein may include additional Nrf2-activating ingredients other than those specifically disclosed herein. In some embodiments, the veterinary supplements disclosed herein may include biologically or pharmacologically active agents useful for treating, inhibiting, and/or preventing conditions or symptoms associated with oxidative stress. For example, in certain specific embodiments, the veterinary supplements disclosed herein include biologically or pharmacologically active agents that treat, inhibit, and/or prevent inflammation, joint disorders, arthritis, cognitive dysfunction, diabetes, pancreatitis, respiratory diseases, and cardiac and vision disorders. In some embodiments, the veterinary supplements disclosed herein include biologically or pharmacologically active agents that support joint function, improve mobility and flexibility, enhance cognitive function, and combinations thereof.

In some embodiments, the veterinary supplements disclosed herein may include other biologically or pharmacologically inactive agents. In certain embodiments, said inactive ingredients include, but are not limited to, binders, diluents, lubricants, glidants, colorants, emulsifiers, disintegrants, starches, water, oils, alcohols, preservatives, sugars, and flavoring agents. In certain specific embodiments, the inactive ingredients are selected from the group consisting of dicalcium phosphate, hydoxypropyl cellulose, hydroxylpropyl methylcellulose, magnesium stearate, maltodextrin, microcrystalline cellulose, silicon dioxide, stearic acid, sucrose fatty acid ester, and combinations thereof. In certain specific embodiments, flavoring agents are selected from the group consisting of chicken flavoring, chicken liver flavoring, smoked bacon flavoring, and combinations thereof.

The present disclosure will be further illustrated by the following non-limiting Examples. These Examples are understood to be exemplary only, and they are not to be construed as limiting the scope of the invention as defined by the appended embodiments.

### EXAMPLES

### Example 1

### A Representative Veterinary Supplement

| ACTIVE INGREDIENT | AMOUNT |
|---|---|
| Nrf2-activating agent: | 150 mg |
| Milk thistle seed extract (Silybum marianum) | (50 mg) |
| Ashwagandha root extract (Withania somnifera) | (33.33 mg) |
| Green tea leaf extract (Camellia sinensis) | (16.67 mg) |
| Bacopa whole herb extract (Bacopa nommieri) | (33.33 mg) |
| Turmeric rhizome extract (Curcuma longa) | (16.67 mg) |
| Omega-3 fatty acids | 200 mg |
| Type II chicken sternum collagen | 125 mg |

Representative dosages for dogs being administered the representative veterinary supplement shown above are as follows:
Toy dogs (4-10 lbs.): ½ tablet daily
Small dogs (11-29 lbs.): 1 tablet daily
Medium dogs (30-69 lbs.): 2 tablets daily
Large dogs (70-109 lbs.): 3 tablets daily
Giant dogs (110+lbs.): 4 tablet daily

### Example 2

### Clinical Trial Conducted with Representative Veterinary Supplement

General Design

A 60-day, double blinded, placebo controlled prospective study in 48 arthritic and 32 healthy client-owned neutered dogs of any breed and gender that were ≧5 years of age and ≧11 lbs.

Materials and Methods

Site Selection: Sites were recruited based on being multi-doctor practices within driving distance of the Monitor, having ultralow temperature storage capacity, and familiar with conducting clinical field trials. Investigators and key personnel were trained in patient data collection methods, sample storage, evaluation forms, and reimbursement policies.

Enrollment Period: This was a time-sensitive study, and as such was initiated under rapid conditions to meet Sponsor deadlines. At Sponsor's request, a healthy group of dogs (n=32) were enrolled in parallel with arthritic dogs (n=48) to reduce timelines and obtain the primary outcomes for assessment. At Sponsor's request, the weights and ages of arthritic dogs were reduced in order to facilitate enrollment to meet timelines.

Patient Selection: Dogs were recruited from a client owned population and selected based on their ability to meet specific inclusion and exclusion criteria. Dogs in the healthy dog cohort were deemed in good health and excluded if receiving any steroids in the last month or starting any new supplements or new diets in the last week. Dogs in the musculoskeletal disability cohort were included if having a musculoskeletal disability (lameness, etc.) consistent with arthritis for at least three months, but excluded if (a) having received a hip or joint replacement surgery, (b) currently receiving NSAIDs, steroids, Adequan, or joint injections, (c) currently on joint supplements such as glucosamine, chondroitin, (d) having any known systemic infection, neuropathology, neoplasia or endocrinopathy, or (e) if starting a joint diet in the last month. Patients were enrolled using a specific patient qualification form.

Owner Selection: Owners were required to sign an informed consent including commitment to the study. Owners that elected to participate received instructions and were scheduled with follow-up appointments by the veterinary hospital.

Cohorts: The Sponsor requested rapid enrollment to obtain TBAR and Catalase data, and therefore the cohorts were shifted to 32 healthy dogs and 48 arthritic dogs to facilitate data capture and reduce clinical phase timelines.

Groupings and Randomization: 80 dogs were randomized in blocks of 8 and allocated into two groups, being Group A(active; n=40) and Group B (placebo; n=40).

Blinding: Investigative site personnel and owners were blinded as to being enrolled into active or placebo groups.

Articles: Test articles (active and placebo) consisted of a chewable flavored tablet administered once daily by weight. The treatment tablet contained the representative veterinary supplement of Example 1. The placebo was an identical product without the anti-oxidant & joint complex. Tablets were provided in a labeled bottle with feeding instructions. The monitor placed a grouping designation “A” or “B” on the product's bottle and cap using a permanent marker. Bottles were placed into boxes labeled “A” or “B”, which corresponded with patient pack assignments.

Patient Packs: Each patient was assigned an individual folder containing all enrollment and evaluation forms. Folders were labeled according to cohort (healthy or arthritic) and grouping (A or B).

Materials: This project required substantial oversight for sample acquisition and maintenance. AHC acquired a large cooler and a shipping cooler for the project. Other materials for the dewars (liquid nitrogen, canes, etc.), folders, cash lockboxes, and other items were also purchased for the study.

Veterinary Evaluations: Veterinarians evaluated arthritic dogs and could evaluate up to 4 joints per animal for lameness, pain and range of motion. The unit of assessment was the dog, and a non-weighted numerical value was assigned to the chosen descriptor. The sum of dog disability was used for assessment of each variable.

Owner Scoring: All owners were issued a questionnaire which addressed overall disability, cognition, energy level, social behavior, and skin and coat assessment, and overall response. Owner disability was scored based on a 0 to 3 scale for walking, running, jumping, rising, lying down, going up or down stairs, squatting, stiffness in morning and evening, ambulation on slippery floors and a willingness to play. The sum of scores was used to calculate an Owner Overall Disability Score. Owners also graded social behavior, cognitive function, energy level, skin condition, coat condition, and overall improvement.

Chemistry and CBC: Whole blood was obtained by venipuncture in 48 dogs at Day 1 and 30 for chemistry and CBC. These data were used for safety evaluation.

Serum: Serum was collected at Days 1, 30 and 60 and stored at -20° C. for future analysis.

Plasma Sample Collection and Storage: Plasma samples were collected from 80 dogs. Whole blood was obtained via venipuncture from clinical subjects on Days 1, 30 and 60 using EDTA purple top tubes for consistency. Whole blood was processed rapidly and lcc of plasma was placed in Nunc Cryotubes suitable for liquid phase liquid nitrogen storage. The Nunc Cryotubes tubes contained 300 mM BHT (butylated hydroxytoluene) in methanol. The addition of BHT to the plasma at collection prevents further oxidation of lipids and other substances in the plasma from storage through the assay. The target final BHT concentration was 3 mM. Samples were flash frozen either on dry ice or in liquid nitrogen and then stored under liquid nitrogen conditions until being shipped for analysis.

Assays: Thiobarbituric Acid Reactive Substances (TBARS) and Catalase Analysis. TBAR analysis was performed using a commercially available TBAR assay (Cayman Chemical Item Number 10009055). Catalase analysis was performed using a commercially available catalase assay (Cayman Chemical Item Number 707002). Samples were rapidly thawed and placed on ice, then assayed using a commercial ELISA reader. Any samples that did not fall on the standard curve were re-assayed at appropriate dilutions (2 dilutions per sample). Catalase data were corrected for volume, incubation time and sample dilution and activity is presented in Units, defined as nmol/min/mL plasma. Lipid peroxidation is measured by the TBARS assay, yielding μM malondialdehyde.

Normalization: TBAR and catalase values were normalized to total protein (TP) of each sample.

Results

Population: 76 out of 80 dogs completed the study (n=44 arthritic; n=32 healthy; 38 female spayed, 36 male neutered, 2 male intact). Average age was 8.6 years old (range 2.5 to 16), average weight was 54.4 lbs (range 13 to 116).

Significant Clinical Findings: Owner Overall Disability score was significantly lower (p=0.027) on average in arthritic dogs receiving active treatment (Group A) vs. placebo. Group A also had a significant improvement (p<0.01) in Owner Overall Improvement at Day 30 but not in placebo.

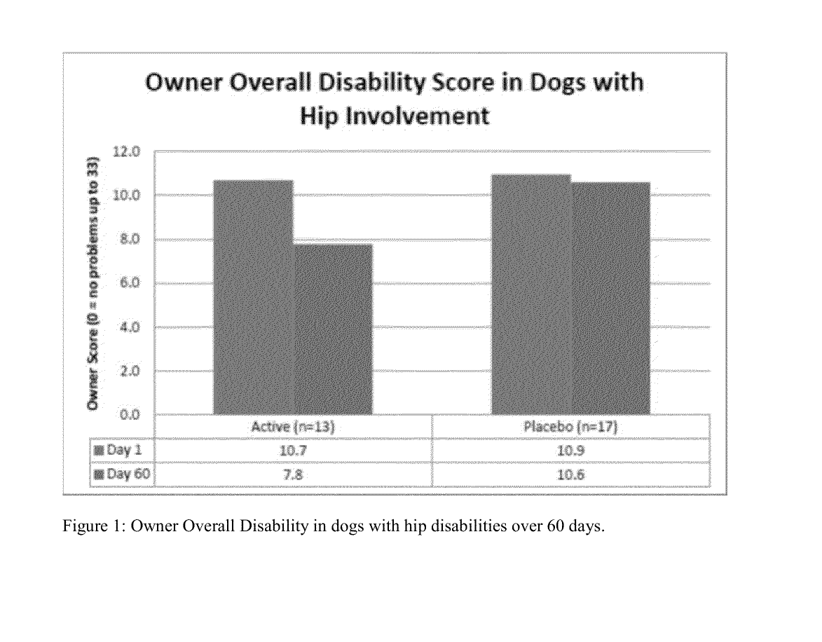

Noteworthy Clinical Findings: Dogs with bilateral hip disability receiving active product had a 27% reduction (improvement) in Owner Overall Disability Scores compared to only a 2% in the placebo group (FIG. 1).

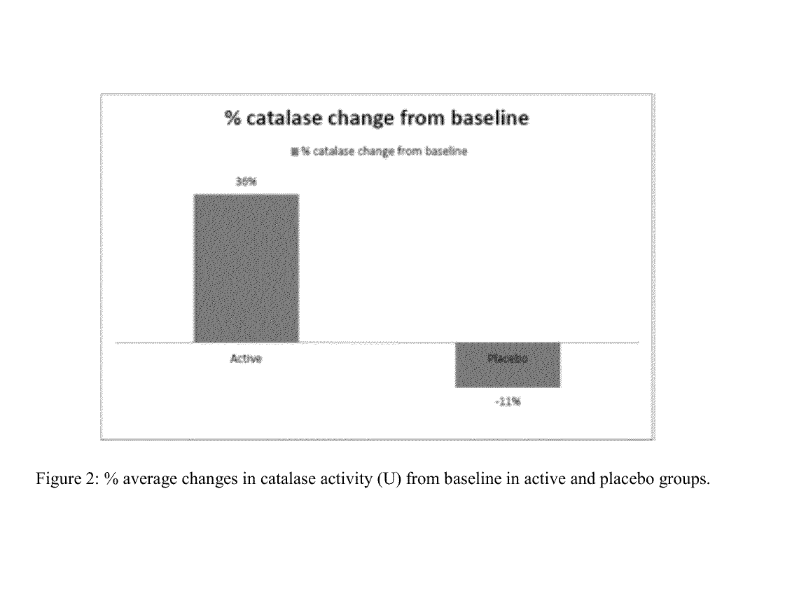

Noteworthy Biochemical Findings: Dogs in the active group had a 47% increase in catalase activity compared to placebo at Day 60. The active group had a 36% increase in catalase from Day 1 to 60, whereas the placebo group had an 11% decrease at Day 60 (FIG. 2).

Correlations: There were no apparent correlations between changes in owner overall disability or veterinary composite changes and weight or age (Day 1 to Day 60 changes).

Chemistry and Complete Blood Count Results: There were no clinically significant changes over time in Groups A or B.

Clinical Safety: Only one dog had a side effect considered to be likely associated with the placebo test article. This patient (5 year old healthy Golden Retriever) developed diarrhea before Day 30, which resolved after discontinuation of the placebo tablet. The diarrhea returned when the dog was placed back on the placebo tablet. The dog's diarrhea resolved without incidence after removal from the study.

Loss to Study: 3 patients did not complete the study, as follows:

Patient 38 (group B): Loss—renal failure—unrelated to product

Patient 55 (group A): Loss—removed owing to failure to control pain

Patient 66 (group A): Loss—euthanized for reasons unrelated to study

TBAR and Catalase Results: There were no significant changes in TBAR or catalase, although the treated group had a strong trend towards an increase in catalase.

### CONCLUSION

Dogs receiving the active product had a 47% difference in catalase activity at Day 60 compared to placebo, as measured by average of all samples. Although not significant, these changes in catalase activity showed a strong trend in the active group, suggesting that the active product may up-regulate oxidative capacity within 60 days.

Owners appeared to be more cognizant of disability changes compared to veterinarians, which is not surprising. One significant finding was the category Owner Overall Disability (Arthritic Dogs), which measured a composite of a variety of everyday disabilities common to arthritic patients. The active treatment showed a significant (p=0.027) averaged (all time-points) overall lower disability compared to the placebo group, as reported by owners. AHC also determined there may be a significant Overall Response to treatment in the active group at Day 30 in arthritic dogs as measured by owners. Additionally, there could be differences in dogs based on which joints are affected.

The study also suggested the product is safe for use in dogs with no evidence of toxicity. No severe adverse events were noted in either group, and no product related adverse events were observed in the treatment group.

1. A veterinary supplement comprising one or more Nrf2-activating agents.

2. The veterinary supplement of claim 1, further comprising omega-3 fatty acids.

3. The veterinary supplement of claim 1, further comprising collagen.

4. The veterinary supplement of claim 1, further comprising omega-3 fatty acids and collagen.

5. The veterinary supplement of claim 1, wherein said one or more Nrf2-activating agents are selected from the group consisting of Bacopa extract, milk thistle extract, Ashwagandha extract, green tea extract, turmeric extract, Gotu kola powder, Aloe vera powder, Gingko biloba leaf extract, N-Acetyl Cysteine, piperine, resveratrol, pterostilbene, sulfurophane, ginger, cinnamon, wasabi, carnosic acid, lipoic acid, licorice, lycopene, and combinations thereof.

6. The veterinary supplement of claim 1, wherein said one or more Nrf2-activating agents are selected from the group consisting of Bacopa extract, milk thistle extract, Ashwagandha extract, green tea extract, turmeric extract, and combinations thereof.

7. The veterinary supplement of claim 4, wherein said one or more Nrf2-activating agents is present in an amount of from about 100 mg to about 200 mg, said omega-3 fatty acids are present in an amount of about 100 mg to about 300 mg, and said collagen is present in an amount of from about 75 mg to about 175 mg.

8. The veterinary supplement of claim 4, wherein said one or more Nrf2-activating agents is present in an amount of from about 125 mg to about 175 mg, said omega-3 fatty acids are present in an amount of about 150 mg to about 250 mg, and said collagen is present in an amount of from about 100 mg to about 150 mg.

9. The veterinary supplement of claim 4, wherein said one or more Nrf2-activating agents is present in an amount of about 150 mg, said omega-3 fatty acids are present in an amount of about 200 mg, and said collagen is present in an amount of about 125 mg.

10. The veterinary supplement of claim 7, wherein: said one or more Nrf2-activating agents comprises Bacopa extract, milk thistle extract, Ashwagandha powder, green tea extract, and turmeric extract.

11. The veterinary supplement of claim 7, wherein said collagen comprises Type II chicken sternum collagen.

12. A veterinary supplement comprising at least about 50 mg milk thistle extract, at least about 33.33 mg Ashwagandha extract, at least about 16.67 mg green tea extract, at least about 33.33 mg B. monniera extract, at least about 16.67 mg Turmeric extract, at least about 200 mg omega-3 fatty acids, and at least about 125 mg of collagen.

13. A veterinary supplement comprising about 50 mg milk thistle extract, about 33.33 mg Ashwagandha extract, about 16.67 mg green tea extract, about 33.33 mg B. monniera extract, about 16.67 mg Turmeric extract, about 200 mg omega-3 fatty acids, and about 125 mg of collagen.

14. The veterinary supplement of claim 1, further comprising inactive ingredients selected from the group consisting of dicalcium phosphate, hydoxypropyl cellulose, hydroxylpropyl methylcellulose, magnesium stearate, maltodextrin, microcrystalline cellulose, silicon dioxide, stearic acid, sucrose fatty acid ester, chicken flavoring, chicken liver flavoring, smoked bacon flavoring, and combinations thereof.

15. A method comprising administering a veterinary supplement of claim 1 to an animal.

16. The method of claim 15, wherein said administration treats, inhibits, reduces, and/or prevents oxidative stress.

17. The method of claim 15, wherein said administration treats, inhibits, reduces, and/or prevents conditions associated with oxidative stress.

18. The method of claim 17, wherein said conditions associated with oxidative stress are selected from the group consisting of inflammation, joint disorders, arthritis, cognitive dysfunction, diabetes, pancreatitis, respiratory diseases, cardiac disorders, vision disorders, and combinations thereof.

19. The method of claim 15, wherein said administration supports joint function, improves mobility and flexibility, enhances cognitive function, and combinations thereof.

20. The veterinary supplement of claim 12, further comprising inactive ingredients selected from the group consisting of dicalcium phosphate, hydoxypropyl cellulose, hydroxylpropyl methylcellulose, magnesium stearate, maltodextrin, microcrystalline cellulose, silicon dioxide, stearic acid, sucrose fatty acid ester, chicken flavoring, chicken liver flavoring, smoked bacon flavoring, and combinations thereof.

21. The veterinary supplement of claim 13, further comprising inactive ingredients selected from the group consisting of dicalcium phosphate, hydoxypropyl cellulose, hydroxylpropyl methylcellulose, magnesium stearate, maltodextrin, microcrystalline cellulose, silicon dioxide, stearic acid, sucrose fatty acid ester, chicken flavoring, chicken liver flavoring, smoked bacon flavoring, and combinations thereof.

22. A method comprising administering a veterinary supplement of claim 12 to an animal.

23. A method comprising administering a veterinary supplement of claim 13 to an animal.
